(19) United States
(12) Patent Application Publication
(10) Pub. No.: US 2022/0163523 A1
(43) Pub. Date: May 26, 2022

(54) TEST STRIP FOR PEANUT IMMUNOFLUORESCENCE ASSAY (IFA), USE THEREOF AND DETECTION METHOD

(71) Applicant: Hangzhou Fudemin Biotechnology Co., Ltd., Hangzhou City (CN)

(72) Inventors: Yong Wu, Hangzhou (CN); Xin Li, Hangzhou (CN); Xuanyi Meng, Hangzhou (CN); Ping Tong, Hangzhou (CN); Juanli Yuan, Hangzhou (CN); Ying Feng, Hangzhou (CN)

(21) Appl. No.: 17/211,535

(22) Filed: Mar. 24, 2021

(30) Foreign Application Priority Data
Nov. 25, 2020 (CN) 202011336762.0

(51) Int. Cl. G01N 33/558; G01N 33/533; C07K 16/16

(52) U.S. Cl. CPC G01N 33/558; C07K 16/16; G01N 33/533

(57) ABSTRACT

The present disclosure provides a test strip for peanut immunofluorescence assay (IFA), use thereof and a detection method, and relates to the technical field of IFA. The test strip of the present disclosure includes a sample pad, a conjugate pad, a nitrocellulose membrane, and a wicking pad arranged successively on a PVC backing card in a left-to-right and end-to-end manner; fluorescent latex microsphere-labeled mixed antibodies are coated on the conjugate pad; anti-Ara h 1 antibody (T1 line), anti-Ara h 2 antibody (T2 line), anti-Ara h 3 antibody (T3 line), anti-total peanut protein (TPP) antibodies (T4 line), and rabbit anti-mouse IgG antibody (C line) are coated on the nitrocellulose membrane, where the T1, T2, T3, and T4 lines are test lines, and the C line is a control line.

### CROSS REFERENCE TO RELATED APPLICATION

This application claims foreign priority to Chinese Patent Application No. 202011336762.0, filed on Nov. 25, 2020, the entire contents of which are incorporated herein by reference.

### TECHNICAL FIELD

The present disclosure relates to the technical field of immunofluorescence assay (IFA), and in particular to a test strip for peanut IFA, use thereof and a detection method.

### BACKGROUND

Peanut is one of the eight food allergens proposed in a report by the Food and Agriculture Organization (FAO) in 1995. Peanut is reportedly a nutritious and well-liked common food, and peanut allergy accounts for 10%-47% of food allergy, which is leading in the eight major foods that produce allergens easily. Compared with other food allergies, peanut allergy has a higher incidence and causes more serious clinical symptoms, attracting wider attention in the fields of public health and food safety.

Peanut allergy can cause allergic enteritis, allergic dermatitis, and other allergic diseases, and even lead to allergic shock and death. There are many cases of allergic death induced by the consumption of peanut foods. Considering that there is no radical cure for such diseases at present, the USA and EU countries require identification of allergen components, such as peanut, on food labels, so as to prevent allergic patients from eating by mistake. Meanwhile, the USA and EU countries begin to spotcheck peanut allergens in some imported food products, so China's food export enterprises often suffer from returns due to incorrect identification of food allergens. Therefore, it is desirable to develop a handy and highly sensitive method for detecting the components and content of peanut allergens.

### SUMMARY

In view of this, an objective of the present disclosure is to provide a test strip for peanut IFA, use thereof and a detection method; the present disclosure enables rapid qualitative and quantitative detection of peanut allergen components in food, featuring easy operation, high accuracy and sensitivity.

To achieve the above purpose, the present disclosure provides the following technical solutions.

The present disclosure provides a test strip for peanut IFA, where the test strip includes a sample pad, a conjugate pad, a nitrocellulose membrane, and a wicking pad arranged successively on a PVC backing card in a left-to-right and end-to-end manner; fluorescent latex microsphere-labeled mixed antibodies are coated on the conjugate pad; the mixed antibodies include: anti-Ara h 1 antibody, anti-Ara h 2 antibody, anti-Ara h 3 antibody, and anti-total peanut protein (TPP) antibodies;

the nitrocellulose membrane includes four test lines and one control line in parallel; the test lines are coated with the anti-Ara h 1 antibody, the anti-Ara h 2 antibody, the anti-Ara h 3 antibody, and the anti-TPP antibodies, respectively; the control line is coated with rabbit anti-mouse IgG antibody; and the antibodies coated on the test lines and the fluorescent latex microsphere-labeled mixed antibodies present an antibody pair.

Preferably, the fluorescent latex microsphere is 50-500 nm in particle size.

Preferably, a method for preparing the conjugate pad coated with the fluorescent latex microsphere-labeled mixed antibodies includes the steps: step a, adsorptively binding fluorescence-labeled streptavidin to latex microspheres to obtain fluorescent latex microspheres;

step b, binding biotin to the mixed antibodies to obtain biotinylated mixed antibodies;

step c, mixing the fluorescent latex microspheres with the biotinylated mixed antibodies to obtain fluorescent latex microsphere-labeled mixed antibodies; and

step d, spraying the fluorescent latex microsphere-labeled mixed antibodies on a conjugate pad;

where there is no temporal relation between steps a and b.

Preferably, a fluorescence marker in step a includes fluorescein isothiocyanate, rhodamine B, tetramethyl rhodamine isothiocynate (TRITC), or fluorescein CY5.

Preferably, during the spraying in step d, the fluorescent latex microsphere-labeled mixed antibodies are sprayed on the conjugate pad in an amount of 2-10 μL/cm.

Preferably, in the mixed antibodies, the anti-Ara h 1 antibody, the anti-Ara h 2 antibody, the anti-Ara h 3 antibody, and the anti-TPP antibodies have a mass ratio of 1:1:1:1.

Preferably, the anti-Ara h 1 antibody, the anti-Ara h 2 antibody, the anti-Ara h 3 antibody, and the anti-TPP antibodies coated on the four test lines have a coating concentration of 0.5-5.0 μL/cm;

the rabbit anti-mouse IgG antibody coated on the control line has a coating concentration of 0.5-5 μL/cm.

The present disclosure further provides use of the above test strip for IFA in the detection of peanut allergen components in food.

Preferably, the peanut allergen components include Ara h1, Ara h2, Ara h3, and TPP.

The present disclosure further provides a method for detecting peanut allergen components in food, including the following steps: dropping 100-120 μL of food solution onto the sample pad of the test strip for IFA, reading fluorescence signals of the test strip after 15 min, and determining the components and content of peanut allergens according to the following standard curve:

$$y = 3.15114 - \frac{3.12726}{1 + {( x / 21. 63173 )}^{1.045 \; 1}} , {R}^{2} = 0.9 \; 997 ;$$

where x is allergen concentration, in IU/mL, and y is fluorescence signal ratio.

The present disclosure provides a test strip for peanut IFA, having a structure as shown in FIG. 1; the test strip includes a sample pad, a conjugate pad, a nitrocellulose membrane, and a wicking pad arranged successively on a PVC backing card in a left-to-right and end-to-end manner; fluorescent latex microsphere-labeled mixed antibodies are coated on the conjugate pad; the mixed antibodies include: anti-Ara h 1 antibody, anti-Ara h 2 antibody, anti-Ara h 3 antibody, and anti-TPP antibodies; the anti-Ara h 1 antibody (T1 line), the anti-Ara h 2 antibody (T2 line), the anti-Ara h 3 antibody (T3 line), the anti-TPP antibodies (T4 line), and rabbit anti-mouse IgG antibody (C line) are coated on the nitrocellulose membrane (NC membrane), where the T1, T2, T3, and T4 lines are test lines, and the C line is a control line. The mixed antibodies of the present disclosure are paired with the antibodies coated on the NC membrane, respectively. Moreover, the mixed antibodies are similar to secondary antibodies, and the antibodies coated on the NC membrane are similar to primary antibodies. The test strip of the present disclosure rapidly and quantitatively detects the type and content of peanut allergens, and features easy operation and high accuracy; recovery rate shall be 90%-110%, with high sensitivity (<0.84 ng/mL).

### BRIEF DESCRIPTION OF THE DRAWINGS

FIG. 1 illustrates the structure of the test strip for IFA provided by the present disclosure;

FIG. 2 illustrates a standard curve for IFA.

### DETAILED DESCRIPTION

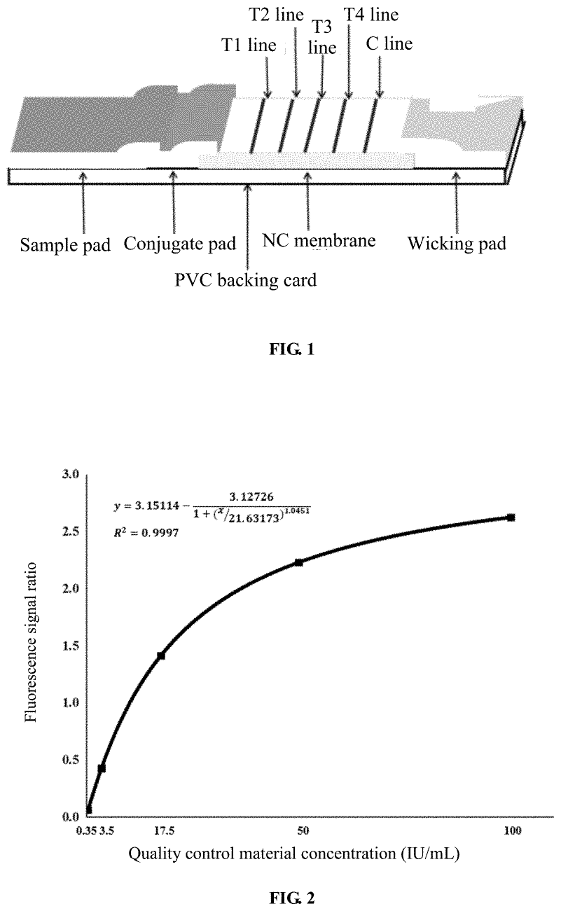

The present disclosure provides a test strip for peanut IFA, having a structure as shown in FIG. 1; the test strip includes a sample pad, a conjugate pad, a nitrocellulose membrane, and a wicking pad arranged successively on a PVC backing card in a left-to-right and end-to-end manner; fluorescent latex microsphere-labeled mixed antibodies are coated on the conjugate pad; the mixed antibodies include: anti-Ara h 1 antibody, anti-Ara h 2 antibody, anti-Ara h 3 antibody, and anti-TPP antibodies.

The nitrocellulose membrane includes four test lines and one control line in parallel; the test lines are coated with the anti-Ara h 1 antibody, the anti-Ara h 2 antibody, the anti-Ara h 3 antibody, and the anti-TPP antibodies, respectively; the control line is coated with rabbit anti-mouse IgG antibody;

The antibodies coated on the test lines and the fluorescent latex micro sphere-labeled mixed antibodies present an antibody pair.

Fluorescent latex microsphere-labeled mixed antibodies are coated on the conjugate pad of the present disclosure. A method for preparing the conjugate pad coated with the fluorescent latex microsphere-labeled mixed antibodies may preferably include the steps: step a, adsorptively binding fluorescence-labeled streptavidin to latex microspheres to obtain fluorescent latex microspheres;

step b, binding biotin to the mixed antibodies to obtain biotinylated mixed antibodies;

step c, mixing the fluorescent latex microspheres with the biotinylated mixed antibodies to obtain fluorescent latex microsphere-labeled mixed antibodies; and

step d, spraying the fluorescent latex microsphere-labeled mixed antibodies on a conjugate pad. There is no temporal relation between steps a and b.

In step a of the present disclosure, the fluorescence-labeled streptavidin and the latex microspheres may preferably have a mass ratio of 1:40; in step b, the biotin and the mixed antibodies may preferably have a volume ratio of 1:4; in step c, the fluorescent latex microspheres and the biotinylated mixed antibodies may preferably have a volume ratio of 10:1. The fluorescent latex microspheres of the present disclosure may preferably be 50-500 nm in particle size. In step a of the present disclosure, a fluorescence marker may preferably include fluorescein isothiocyanate, rhodamine B, tetramethyl rhodamine isothiocynate (TRITC), or fluorescein CY5. In the mixed antibodies of the present disclosure, the anti-Ara h 1 antibody, the anti-Ara h 2 antibody, the anti-Ara h 3 antibody, and the anti-TPP antibodies may preferably have a mass ratio of 1:1:1:1. Sources of the anti-Ara h 1 antibody, the anti-Ara h 2 antibody, the anti-Ara h 3 antibody, and the anti-TPP antibodies are not particularly limited in the present disclosure, as long as commercially available antibodies conventional in the art may preferably be selected. In step d of the present disclosure, the fluorescent latex microsphere-labeled mixed antibodies may preferably be sprayed on the conjugate pad in an amount of 2-10 μL/cm. In the present disclosure, all of the fluorescent latex microsphere-labeled mixed antibodies may be secondary antibodies.

The nitrocellulose membrane (NC membrane) of the present disclosure includes four test lines and one control line in parallel; the test lines are coated with the anti-Ara h 1 antibody, the anti-Ara h 2 antibody, the anti-Ara h 3 antibody, and the anti-TPP antibodies, respectively; the control line is coated with rabbit anti-mouse IgG antibody. Methods for preparing the NC membrane are not particularly limited in the present disclosure, preferably including steps of: diluting the anti-Ara h 1 antibody, the anti-Ara h 2 antibody, the anti-Ara h 3 antibody, the anti-TPP antibodies, and the rabbit anti-mouse IgG antibody with coating buffer, respectively; streaking five diluted antibodies on the NC membrane in parallel, respectively; after permeation of the antibodies into the NC membrane, forming test lines coated with the anti-Ara h 1 antibody, the anti-Ara h 2 antibody, the anti-Ara h 3 antibody, and the anti-TPP antibodies and a control line coated with the rabbit anti-mouse IgG antibody, respectively. The anti-Ara h 1 antibody, the anti-Ara h 2 antibody, the anti-Ara h 3 antibody, and the anti-TPP antibodies coated on the four test lines of the present disclosure may have a coating concentration of 0.5-5.0 μL/cm; the rabbit anti-mouse IgG antibody coated on the control line may have a coating concentration of 0.5-5 μL/cm. In the present disclosure, in view of different operations of the test and control lines during streaking, when streaking the test lines, the anti-Ara h 1 antibody, the anti-Ara h 2 antibody, the anti-Ara h 3 antibody, and the anti-TPP antibodies may preferably be streaked successively at a concentration of 3 mg/mL (liquid output of peristaltic pump 0.4 mL/min, streaking speed 50 m/20 min) and blast-dried in a drying oven at 20° C. for 12 h. When streaking the control line, the rabbit anti-mouse IgG antibody may preferably be streaked on the NC membrane at a concentration of 8 mg/mL (liquid output of peristaltic pump 0.4 mL/min, streaking speed 50 m/20 min); the line is parallel to the test lines and blast-dried in the drying oven at 20° C. for 12 h.

Preferably, in the present disclosure, the sample pad, the conjugate pad, the NC membrane, and the wicking pad may be assembled and pasted on the PVC backing card, and cut into test strips as shown in FIG. 1 on a slitter as required (4 mm). The test strip of the present disclosure may be mass produced, suitable for rapid clinical diagnosis and on-site rapid detection, and easy to store.

The present disclosure provides use of the above test strip for IFA in the detection of peanut allergen components in food.

The peanut allergen components of the present disclosure may preferably include Ara h1, Ara h2, Ara h3, and TPP, where the Ara h1, the Ara h2, and the Ara h3 may not be involved in the TPP.

The present disclosure further provides a method for detecting peanut allergen components in food, including the following steps: dropping 100-120 μL of food solution onto a sample pad of the above test strip for IFA, reading fluorescence signals of the test strip after 15 min, and determining the components and content of peanut allergens according to the following standard curve:

$$y = 3.15114 - \frac{3.12726}{1 + {( x / 21. 63173 )}^{1.045 \; 1}} , {R}^{2} = 0.9997 ;$$

where x is allergen concentration, in IU/mL, and y is fluorescence signal ratio.

In the present disclosure, after a test sample is dropped onto the sample pad, the sample reacts with and binds to the mixed antibodies on the conjugate pad, reacts with the anti-Ara h 1 antibody, the anti-Ara h 2 antibody, the anti-Ara h 3 antibody, and the anti-TPP antibodies in a test zone successively, and finally reaches a control zone to end the reactions.

The test strip for peanut IFA, the use thereof and the detection method provided by the present disclosure will be described in detail below in conjunction with examples, but they should not be construed as limiting the protection scope of the present disclosure.

All reagents used in the present disclosure, unless specified otherwise, may be commercially available products, where:

anti-Ara h 1 antibody: Indoor Biotechnologies (Cat#: MA-2F7-1 and MA-5C2-1),

anti-Ara h 2 antibody: Indoor Biotechnologies (Cat#: MA-2H3-1 and MA-1C4-1),

anti-Ara h 3 antibody: Indoor Biotechnologies (Cat#: MA-1E8-1 and MA-4G9-1), and

anti-TPP antibodies: Indoor Biotechnologies (Cat#: MA-3B8-1).

### EXAMPLE 1

1. Preparation of Conjugate Pad

1) Preparation of Fluorescent Latex Microspheres

Preparation of fluorescent latex microspheres: Adsorption buffer (50 mM, pH 5.8 citrate buffer) was used to dilute latex microspheres with a particle size of 400 nm to obtain 6 mL of latex microsphere suspension with a final concentration of 30 mg/mL; appropriate red fluorescein rhodamine-labeled streptavidin was charged into the adsorption buffer, with a final volume of 6 mL; the above latex microsphere suspension was charged into the above adsorption buffer with red fluorescein rhodamine-labeled streptavidin to obtain a mixture; the resulting mixture was incubated for 1-2 h at room temperature while constantly stirring, followed by centrifugation; a precipitate was collected, dissolved in storage buffer (adsorption buffer with 0.06% bovine serum albumin (BSA)), and stored at 4° C. for use.

2) Preparation of Biotinylated Anti-Peanut Mixed Antibodies

Anti-Ara h 1 antibody, anti-Ara h 2 antibody, anti-Ara h 3 antibody, and anti-TPP antibodies were mixed in a mass ratio of 1:1:1:1 (mixed antibodies, M1) and diluted to 3 mL with 0.2 M pH 4.7 sodium acetate buffer, and anti-peanut antibody M1 was fully dialyzed with 0.2 M pH 4.7 sodium acetate buffer alternatively; 1 mL of N-hydroxysuccinimidobiotin (NHSB) was dissolved in 1 mL of dimethylsulfoxide (DMSO) to obtain an NHSB solution; 25 μL of NHSB was charged into the above 3 mL of anti-peanut antibody Ml, stirred for 2-4 h, continuously stirred for 10 min at room temperature, and dialyzed with 20 mM, pH 3.9 phosphate buffer saline (PBS) to obtain biotinylated mixed antibodies M1.

3) Preparation of Fluorescent Latex Microsphere-Labeled Anti-Peanut Antibody

The fluorescent latex microspheres obtained in step 1) and the biotinylated mixed antibodies obtained in step 2) were mixed in a volume ratio of 10:1, and centrifuged after reacting for 30 min; a precipitate was dissolved in storage buffer, followed by restoring the original volume.

4) The Fluorescent Latex Microsphere-Labeled Anti-Peanut Mixed Antibodies were Sprayed on the Conjugate Pad in an Amount of 2-10 μL/cm.

2. Preparation of NC Membrane

1) Membrane treatment: An NC membrane was marked and immersed in pH membrane treatment buffer (TBS) for 5-10 min.

2) A sample applicator was assembled; an immersed NC membrane was placed on a lay-flat pad, and an antibody application plate was arranged, leaving room for labeling thereon.

3) Preparation of anti-peanut antibody test zone: The anti-Ara h 1 antibody, the anti-Ara h 2 antibody, the anti-Ara h 3 antibody, and the anti-TPP antibodies were streaked successively at a concentration of 3 mg/mL (liquid output of peristaltic pump 0.4 mL/min, streaking speed 50 m/20 min) and blast-dried in a drying oven at 20° C. for 12 h.

4) Preparation of control zone: The rabbit anti-mouse IgG antibody was streaked on the NC membrane 4 at a concentration of 8 mg/mL (liquid output of peristaltic pump 0.4 mL/min, streaking speed 50 m/20 min); the line was parallel to lines in the test zone and blast-dried in the drying oven at 20° C. for 12 h.

5) The above NC membrane was blocked with blocking buffer (prepared from 100 mL of PBS and 0.5 g of BSA) for 60 min at 37° C., removed, dried for 2 h at 37° C., and sealed in a bag for use.

6) Assembly of test strip

A sample pad, a conjugate pad, an NC membrane, and a wicking pad were assembled and pasted on a PVC backing card, and cut into test strips as shown in FIG. 1 on a slitter as required (4 mm).

3. Detection of Antigen to be Tested

After 100-120 μL of test sample was dropped onto the sample pad, the sample reacted with and bound to the anti-peanut mixed antibodies on the conjugate pad, reacted with the anti-Ara h 1 antibody, the anti-Ara h 2 antibody, the anti-Ara h 3 antibody, and the anti-TPP antibodies in the test zone successively, and finally reached the control zone to end the reactions; the test strip was placed on a specific fluorescence microplate reader, and fluorescence signal intensity was read for quantitative determination.

The linearity of dose-response curve: Serial calibration solutions prepared from a calibrator in a kit were determined; concentrations were 100.00, 50.00, 17.50, 3.50, and 0.35 IU/mL, respectively, and fitted in a double logarithmic model or other appropriate mathematical models. The model fitting result should be consistent with the following: inter-run precision (CV%) should be ≤10.0%; between-run precision (CV %) should be ≤15.0%.

The linear analysis of the dose-response curve concluded that: within the range of 0.35-100 IU/mL, there was a smooth increasing curve of each concentration versus measured value, and the lower limit of detection (a minimum concentration detected by a test system with CV<15%) was 0.35 IU/mL. The curve equation is shown in FIG. 2:

$$y = 3.15114 - \frac{3.12726}{1 + {( x / 21. 63173 )}^{1.045 \; 1}} , {R}^{2} = 0.9997 ;$$

where x is allergen concentration, in IU/mL, and y is fluorescence signal ratio.

The foregoing description is merely a preferred example of the present disclosure; it should be noted that several improvements and modifications can also be made by those of ordinary skill in the art without departing from the principles of the present disclosure, and these improvements and modifications should also be regarded as the protection scope of the present disclosure.

1. A test strip for peanut immunofluorescence assay (IFA), wherein the test strip comprises a sample pad, a conjugate pad, a nitrocellulose membrane, and a wicking pad arranged successively on a PVC backing card in a left-to-right and end-to-end manner;
fluorescent latex microsphere-labeled mixed antibodies are coated on the conjugate pad; the mixed antibodies comprise: anti-Ara h 1 antibody, anti-Ara h 2 antibody, anti-Ara h 3 antibody, and anti-total peanut protein (TPP) antibodies;
the nitrocellulose membrane comprises four test lines and one control line in parallel; the test lines are coated with the anti-Ara h 1 antibody, the anti-Ara h 2 antibody, the anti-Ara h 3 antibody, and the anti-TPP antibodies, respectively; the control line is coated with rabbit anti-mouse IgG antibody; and
the antibodies coated on the test lines and the fluorescent latex microsphere-labeled mixed antibodies present an antibody pair.

2. The test strip for IFA according to claim 1, wherein the fluorescent latex microsphere is 50-500 nm in particle size.

3. The test strip for IFA according to claim 1, wherein a method for preparing the conjugate pad coated with the fluorescent latex microsphere-labeled mixed antibodies comprises the steps:
step a, adsorptively binding fluorescence-labeled streptavidin to latex microspheres to obtain fluorescent latex microspheres;
step b, binding biotin to the mixed antibodies to obtain biotinylated mixed antibodies;
step c, mixing the fluorescent latex microspheres with the biotinylated mixed antibodies to obtain fluorescent latex microsphere-labeled mixed antibodies; and
step d, spraying the fluorescent latex microsphere-labeled mixed antibodies on a conjugate pad;
wherein there is no temporal relation between steps a and b.

4. The test strip for IFA according to claim 3, wherein a fluorescence marker in step a comprises fluorescein isothiocyanate, rhodamine B, tetramethyl rhodamine isothiocynate (TRITC), or fluorescein CYS.

5. The test strip for IFA according to claim 3, wherein during the spraying in step d, the fluorescent latex microsphere-labeled mixed antibodies are sprayed on the conjugate pad in an amount of 2-10 μL/cm.

6. The test strip for IFA according to claim 1, wherein in the mixed antibodies, the anti-Ara h 1 antibody, the anti-Ara h 2 antibody, the anti-Ara h 3 antibody, and the anti-TPP antibodies have a mass ratio of 1:1:1:1.

7. The test strip for IFA according to claim 1, wherein the anti-Ara h 1 antibody, the anti-Ara h 2 antibody, the anti-Ara h 3 antibody, and the anti-TPP antibodies coated on the four test lines have a coating concentration of 0.5-5.0 μL/cm;
the rabbit anti-mouse IgG antibody coated on the control line has a coating concentration of 0.5-5 μL/cm.

8. Use of the test strip for IFA according to claim 1 in the detection of peanut allergen components in food.

9. The use according to claim 8, wherein the peanut allergen components comprise Ara h1, Ara h2, Ara h3, and TPP.

10. A method for detecting peanut allergen components in food, comprising the following steps: dropping 100-120 μL of food solution onto the sample pad of the test strip for IFA according to claim 1, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

11. Use of the test strip for IFA according to claim 2 in the detection of peanut allergen components in food.

12. The test strip for IFA according to claim 3, wherein in the mixed antibodies, the anti-Ara h 1 antibody, the anti-Ara h 2 antibody, the anti-Ara h 3 antibody, and the anti-TPP antibodies have a mass ratio of 1:1:1:1.

13. Use of the test strip for IFA according to claim 3 in the detection of peanut allergen components in food.

14. A method for detecting peanut allergen components in food, comprising the following steps: dropping 100-120 μL of food solution onto the sample pad of the test strip for IFA according to claim 2, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

15. A method for detecting peanut allergen components in food, comprising the following steps: dropping 100-120 μL of food solution onto the sample pad of the test strip for IFA according to claim 3, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

16. A method for detecting peanut allergen components in food, comprising the following steps: dropping 100-120 μL of food solution onto the sample pad of the test strip for IFA according to claim 4, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

17. A method for detecting peanut allergen components in food, comprising the following steps: dropping 100-120 μL of food solution onto the sample pad of the test strip for IFA according to claim 5, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

18. A method for detecting peanut allergen components in food, comprising the following steps: dropping 100-120 μL of food solution onto the sample pad of the test strip for IFA according to claim 6, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

19. A method for detecting peanut allergen components in food, comprising the following steps: dropping 100-120 μL of food solution onto the sample pad of the test strip for IFA according to claim 7, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.

20. A method for detecting peanut allergen components in food, comprising the following steps: dropping 100-120 μL of food solution onto the sample pad of the test strip for IFA according to claim 12, reading fluorescence signals of the test strip after 15 min, and determining the components and content of peanut allergens according to the following standard curve:
$$y = 3.15114 - \frac{3.12726}{1 + {( x / 21. 63173 )}^{1.045 \; 1}} , {R}^{2} = 0.9 \; 997 ;$$
wherein x is allergen concentration, in IU/mL, and y is fluorescence signal ratio.
